(19) United States
(12) Patent Application Publication
(10) Pub. No.: US 2025/0020554 A1
(43) Pub. Date: Jan. 16, 2025

(54) METHODS FOR SCREENING FOR, OR DIAGNOSIS OF, ENDOMETRIOSIS AND METHODS OF PREPARING CELLS THEREFOR

(71) Applicant: DIAGNOSTRIX AS, Sandnes (NO)

(72) Inventor: Dorothee GOLDMAN, Hammondsport, NY (US)

(21) Appl. No.: 18/708,560

(22) PCT Filed: Nov. 9, 2022
(86) PCT No.: PCT/EP2022/081362

§ 371 Date: May 8, 2024

Related U.S. Application Data
(60) Provisional application No. 63/277,342, filed on Nov. 9, 2021.

(51) Int. Cl. G01N 1/30; G01N 33/50

(52) U.S. Cl. CPC G01N 1/30; G01N 33/5091; G01N 2800/364

(57) ABSTRACT

The invention relates to an in vitro method for preparing cells such that they are suitable for use in screening for, or diagnosis of, endometriosis in a human female subject. The method comprises sequentially treating mucosal epithelial cells which have been obtained from the female subject and immobilised on a transparent substrate with i) a flavonol pigment and ii) an anthocyanidin mono-glycoside pigment which has at least two OH groups on the B ring of the anthocyanidin, in that order. The invention is also directed to a treated cell prepared by the preparation method of the invention and kits for carrying out said method. The invention also provides in vitro methods of screening for, or diagnosing, endometriosis in a human female subject.

### TECHNICAL FIELD

The invention relates to an in vitro method for preparing cells such that they are suitable for use in screening for, or diagnosis of, endometriosis in a human female subject. The invention is also directed to a treated cell prepared by the preparation method of the invention and kits for carrying out said method. The invention also provides in vitro methods of screening for, or diagnosing, endometriosis in a human female subject.

### BACKGROUND

Endometriosis is a disorder characterised by ectopic lesions of endometrial tissue in various organs of the body outside the uterine cavity. Ectopic lesions are typically found on the ovaries, fallopian tubes, and ligaments that support the uterus, areas around the vagina and the uterus, and areas within the peritoneal and pelvic cavities. The ectopic lesions form benign tumours on organs which can lead to inflammation, severe discomfort, pelvic pain and reproductive failure.

Endometriosis is usually diagnosed via laparoscopy of the pelvic cavity, which is a costly and invasive procedure. Less invasive procedures for diagnosis of endometriosis include ultrasound or magnetic resonance imaging (MRI) but these are still costly and require significant technical expertise during both the imaging and interpretation stages.

U.S. Pat. No. 5,922,613 discloses a method for screening for endometriosis comprising contacting a saliva sample from a female human subject with an anthocyanin pigment that is a diglucoside.

U.S. Pat. No. 8,841,130 discloses an endometriosis screening method comprising contacting a saliva sample obtained from a female mammal during the luteal phase of a menstrual cycle with an apatite compound, a flavonol pigment and an anthocyanin pigment. This document discloses that the apatite compound leads to aggregation and precipitation of saliva protein complexes that are specific to women with endometriosis and that this aggregation and precipitation leads to the sample having a cloudy appearance. It is also disclosed that the treatment with the flavonol and anthocyanin leads to a colour response in the sample which is indicative of endometriosis. The anthocyanin pigments disclosed in U.S. Pat. No. 8,841,130 B2 are di-glycosides.

U.S. Pat. No. 8,722,349 B2 also discloses endometriosis screening methods. One method comprises providing a mixture containing a bodily sample from a female subject and a flavonoid pigment, which may be either quercetin or an anthocyanin, measuring the change in optical density values of the mixture at two wavelengths, evaluating the rate of change in absorbency values between two wavelengths and comparing this rate of change to a reference scale. A further method comprises mixing a bodily fluid from a female subject with a flavonoid pigment, which is preferably quercetin, and treating the sample with an indicator, which is preferably iodine, to produce a colour response.

U.S. Pat. Nos. 5,922,613, 8,841,130 B2 and 8,722,349 B2 do not disclose application of an anthocyanidin mono-glycoside pigment, either alone or after application of a flavonol pigment. Moreover, these documents do not disclose that observing whether or not nuclei of mucosal epithelial cells appear to be stained following treatment with a flavonol pigment and an anthocyanidin mono-glycoside pigment in accordance with the present invention can be used to screen for endometriosis.

There remains a need for low cost, accurate and non-invasive methods for screening for and diagnosing endometriosis.

### SUMMARY OF THE INVENTION

A first aspect of the invention provides an in vitro method for preparing mucosal epithelial cells such that they are suitable for use in screening for, or diagnosis of, endometriosis in a human female subject, the method comprising:
sequentially treating mucosal epithelial cells which have been obtained from the human female subject and immobilised on a transparent substrate with i) a flavonol pigment and ii) an anthocyanidin mono-glycoside pigment which has at least two OH groups on the B ring of the anthocyanidin, in that order. The method of this first aspect is referred to herein as “preparation method A”.

A second aspect of the invention provides a treated mucosal epithelial cell obtained by, or obtainable by, the method of the first aspect.

A third aspect provides an in vitro method of screening for, or diagnosing, endometriosis in a human female subject, the method comprising:
observing whether or not the nuclei of one or more mucosal epithelial cells which have been obtained from the human female subject and immobilised on a transparent substrate appear to be darker than the surrounding part(s) of the cell(s) (such as the cell surface) following sequential treatment with i) a flavonol pigment and ii) an anthocyanidin mono-glycoside pigment which has at least two OH groups on the B ring of the anthocyanidin, in that order. This method is referred to herein as “screening/diagnosis method A”.

A fourth aspect provides an in vitro method of screening for, or diagnosing, endometriosis in a human female subject, the method comprising:
sequentially treating mucosal epithelial cells which have been obtained from the human female subject and immobilised on a transparent substrate with i) a flavonol pigment and ii) an anthocyanidin mono-glycoside pigment which has at least two OH groups on the B ring of the anthocyanidin, in that order, and
observing whether or not the nuclei of one or more of the cells appear to be darker than the surrounding part(s) of the cell(s) (such as the cell surface). This method is referred to herein as “screening/diagnosis method B”.

In the methods of the invention, the cells are preferably observed using bright-field (BF) microscopy.

A fifth aspect provides a kit for preparing samples for screening for, or diagnosing, endometriosis comprising a flavonol pigment and an anthocyanidin mono-glycoside pigment which has at least two OH groups on the B ring of the anthocyanidin. The kit preferably comprises instructions to direct the user to carry out a method of the invention, such as preparation method A of the invention.

As set out below, following treatment with the pigments in accordance with the invention, the cell nuclei appear darker than the surrounding part(s) of the cell (such as the cytoplasm or cell surface) when the cells are from a subject who does not have endometriosis. In other words, the cell nuclei appear to be stained (i.e. have higher intensity of colour than the rest of the cell) when the subject does not have endometriosis.

In contrast, the cell nuclei do not appear darker than the surrounding part(s) of the cell when the cells are from a subject who has endometriosis. Some low level background staining of the cell may occur when the patient has endometriosis, but the nucleus does not appear darker than the surrounding part(s) of the cell (such as the cytoplasm or the cell surface), and may even appear somewhat paler. Often, cells (including the nuclei) from a subject who has endometriosis do not show any colouration following the pigment treatment.

Furthermore, the inventor has observed that the background staining of cells from subjects who have endometriosis may fade within approximately 1-2 hours after treatment. However, following treatment with the pigments in accordance with the invention, cell nuclei from subjects who do not have endometriosis typically continue to appear darker than the surrounding part(s) of the cell, such as the cytoplasm or cell surface, over at least this time period, and usually for significantly longer time periods such as several days or weeks after being exposed to the pigment treatment.

To the extent that they are combinable, features disclosed in relation to one aspect of the invention are explicitly disclosed in combination with each of the other aspects of the invention.

### BRIEF DESCRIPTION OF THE FIGURES

FIG. 1 is a schematic illustration of screening/diagnosis methods A and B.

FIGS. 2A and 2B are bright-field (BF) optical microscope photographs of buccal cells obtained by swabbing the inside of the cheeks of female subjects which were treated with a comparative pigment methylene blue. The cell nuclei appeared darker than the surrounding parts of the cell in cells from both a subject who was known to have endometriosis (FIG. 2A) and a subject who was known not to have endometriosis (FIG. 2B).

FIG. 3 shows BF optical microscope photographs of buccal cells obtained by swabbing the inside of the cheeks of female subjects which were then treated with quercetin followed by malvidin-3,5-diglucoside in a comparative experiment. All of FIGS. 3A-3C show cells from subjects who were known not have endometriosis and the cell nuclei do not appear darker than the surrounding parts of the cell.

FIGS. 4A-4C and 5 are BF optical microscope photographs of buccal cells obtained by swabbing the inside of the cheeks of female subjects who were known not to have endometriosis which were treated with quercetin followed by cyanidin-3-glucoside. The cell nuclei appear darker than the surrounding parts of the cell.

FIGS. 6A-6D, 7 and 8 are BF optical microscope photographs of buccal cells obtained by swabbing the inside of the cheeks of female subjects who were known to have endometriosis which were treated with quercetin followed by cyanidin-3-glucoside. The cell nuclei do not appear darker than the surrounding parts of the cell. Also shown in FIG. 7A are dark spots which were artefacts formed outside the cells during sample preparation. These artefacts are not diagnostic of the presence or absence of endometriosis.

### DETAILED DESCRIPTION

The invention provides a low cost and non-invasive in vitro method of screening for, or diagnosing, endometriosis that does not require specialist medical expertise to carry out.

In particular, it has unexpectedly been found that there is a difference in apparent nuclear staining observed in mucosal epithelial cells from female subjects that have endometriosis and in mucosal epithelial cells from female subjects that do not have endometriosis following treatment of the cells sequentially with i) a flavonol pigment and ii) an anthocyanidin mono-glycoside pigment which has at least two OH groups on the B ring of the anthocyanidin, in that order.

Preferably, the cells are collected from a subject during the follicular phase of their menstrual cycle, more preferably during the early follicular phase. Prior to the treatment, the cells have been immobilised on a transparent substrate.

As used herein, immobilised cells are cells that have been dried (e.g. allowed to air dry) on the transparent substrate. The cells may be air dried under ambient conditions, preferably for about 10-30 minutes and typically for at least about 20 minutes. As used herein, ambient conditions means room temperature and pressure (approximately 18-25° C. and approximately 1 atmosphere [101 kPa] of pressure).

As used herein, the term “surrounding part(s) of the cell” refers to extra-nuclear parts of the cell such as the cell surface and/or the cytoplasm, preferably it refers to the cell surface.

Following treatment with the pigments in accordance with the invention, the cell nuclei appear darker than the surrounding part(s) of the cell (such as the cell surface) when the cells are from a subject who does not have endometriosis. In other words, the cell nuclei appear to be stained when the subject does not have endometriosis.

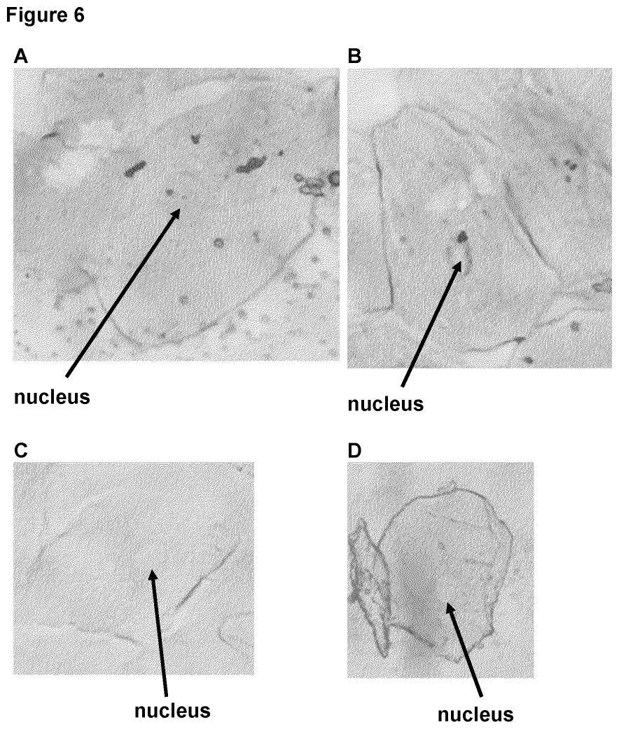

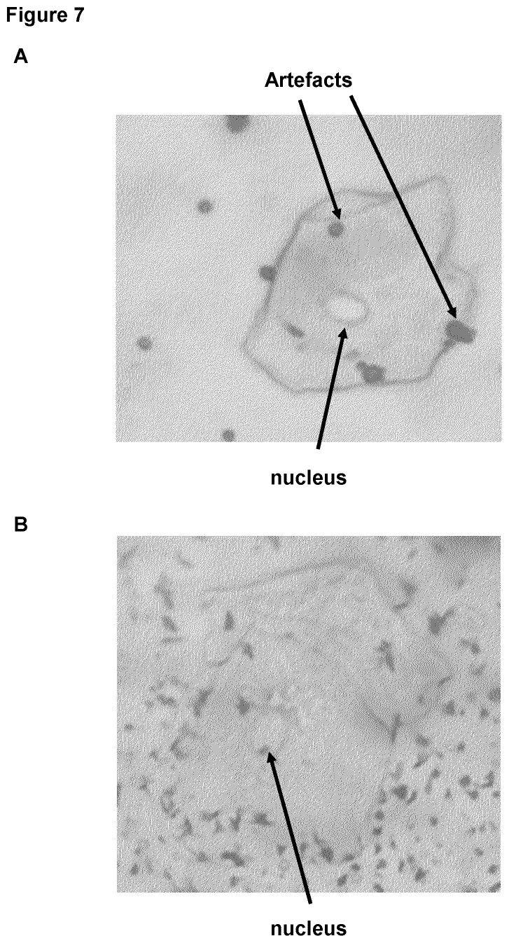

In contrast, the cell nuclei do not appear darker than the surrounding part(s) of the cell when the cells are from a subject who has endometriosis. Some low level background staining of the cell may occur when the patient has endometriosis but the nucleus does not appear darker than the surrounding part(s) of the cell (such as the cytoplasm or cell surface). Following treatment with the pigments in accordance with the invention, the cell nuclei may appear paler (i.e. of lower colour intensity or without colour) than does the rest of the cell (see FIG. 6B and FIG. 7).

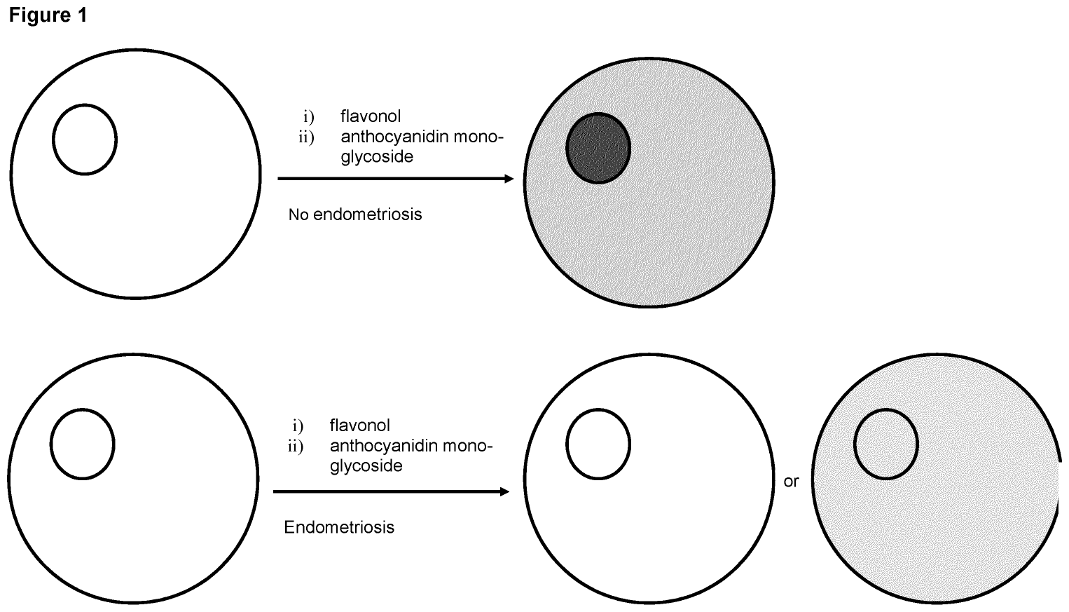

As illustrated schematically in FIG. 1, treating mucosal epithelial cells sequentially with i) a flavonol pigment and ii) an anthocyanidin mono-glycoside pigment which has at least two OH groups on the B ring of the anthocyanidin, in that order, leads to a darker appearance of the cell nuclei compared to the surrounding part(s) of the cell, such as the cytoplasm or cell surface, when a subject does not have endometriosis. However, when a subject has endometriosis, the nuclei do not appear darker than the surrounding part(s) of the cell, such as the cytoplasm or cell surface. For example, no parts of the cell (including the nucleus) may show coloration or both the nucleus and the rest of the cell may show some low level coloration but the nucleus may appear the same colour as the rest of the cell. As illustrated in FIG. 1, some background staining of the surrounding part(s) of the cell, such as the cytoplasm or cell surface, may occur whether or not a subject has endometriosis.

Furthermore, the inventor has observed that any staining of cells from subjects who have endometriosis generally fades within approximately 1-2 hours after treatment. However, following treatment with the pigments in accordance with the invention, cell nuclei from subjects who do not have endometriosis typically continue to appear darker than the surrounding part(s) of the cell, such as the cytoplasm or cell surface, over at least this time period and usually for significantly longer time periods, such as for several days, weeks or months after the staining procedure has been performed. For example, the cell nuclei may continue to appear darker for months after the treatment with the pigments, such as up to 3-9 months after the treatment with the pigments. This behaviour can be used to help confirm any initial assessment as to whether the cells are from a subject with or without endometriosis.

### Subjects

Endometriosis is a human aliment and the methods of the invention are therefore carried out on cells obtained from human female subjects. The subject should have experienced a menstrual cycle within the previous 90 days prior to the test. The methods are not therefore applicable to cells from post-menopausal women.

Methods of the invention comprise treating suitable cells that have been previously obtained from a human female subject and immobilised on a transparent substrate with the flavonol pigment and the anthocyanidin mono-glycoside pigment. That is, the cells have already been obtained from the subject and it is not necessary as part of the methods to obtain the cells from the subject. Similarly, the cells have been immobilised on the transparent substrate and so it is not necessary as part of the methods to immobilise the cells on the transparent substrate.

However, in some cases, preparation method A or screening/diagnosis methods A or B may further comprise a step of obtaining the cells from the subject and/or immobilising the cells on a transparent substrate.

It is preferred that the cells are collected from the subject in the follicular phase of the menstrual cycle, more preferably the early follicular phase, but not when LH (luteinizing hormone) is elevated such as during the ovulatory phase.

Cells from women with no endometriosis show higher colour intensity of nuclear staining when the cells are collected in the follicular phase, particularly the early follicular phase.

However, whether or not a woman has endometriosis, cell nuclei will not appear darker than the surrounding part(s) of the cell following treatment with the pigments if the cells are collected at the time of ovulation when levels of luteinizing hormone (LH) are elevated. Elevated levels of LH may be above 20 IU/L in urine (wherein one NIBSC 96/602 ampoule contains 189 IU of LH). To control for this, it is advisable for subjects to take a urine ovulation test to monitor LH levels before collecting cells for use in the methods of the invention. Urine tests to monitor ovulation via LH levels are commercially available. If an ovulation test is positive, cells should not be collected for testing according to the methods of the invention. The ovulation test should be repeated until LH levels decrease (i.e. when the ovulation test is no longer positive), after which cells may be collected for testing according to the present invention. Blood tests for checking levels of LH and/or whether the patient is in the ovulatory phase may also be used.

Preferably, the cells have been obtained from the subject during the follicular phase of the menstrual cycle, more preferably during the early follicular phase. The early follicular phase is defined herein as day 1 to day 8 of the of the subject's menstrual cycle, wherein the first day of menstrual bleeding is considered day 1 of the menstrual cycle.

As set out above, whether or not a woman has endometriosis, cell nuclei will not appear darker than the surrounding part(s) of the cell following treatment with the pigments if the cells are collected when levels of luteinizing hormone (LH) are elevated. Such false positives are more likely to be avoided if the cells have been obtained from the subject during the early follicular phase. The ability to differentiate between endometriosis and no endometriosis using the method of the invention has been found to have the highest specificity if the cells are collected during the early follicular phase.

### Cells and Cell Collection

The methods of the invention are carried out using mucosal epithelial cells. These may be cells from the mouth, cervix or vagina of a subject. A sample containing cells may be obtained from the lining of the vagina, cervix or oral cavity, preferably from the oral cavity. The cells are preferably obtained by swabbing one of these areas. For example, cells may be obtained, preferably by swabbing, the inside of the cheek, the soft palate or gingival areas of the mouth, preferably the soft palate. The cells may be present in a saliva sample. In some cases, a saliva sample may be applied to the transparent substrate in the methods of invention. When collecting oral mucosal cells (including if a saliva sample is used), the subjects should refrain from eating or drinking in the 30 minutes prior to the cell collection.

Any conventional swab may be used to obtain the cells. For example, a cellulose stick may be used as the swab.

Once obtained, the cells are then immobilised on a transparent substrate. For example, the cells may be transferred to the substrate by rubbing the swab on the substrate, optionally in one direction. A particularly suitable transparent substrate is a glass or clear plastic slide, such as acrylic.

The inventor has observed that the difference in nuclear staining between cells from subjects who have and do not have endometriosis is more apparent when using hydrophobic transparent substrates. In the invention, it is therefore preferred to use a hydrophobic transparent substrate. As used herein, a substrate is hydrophobic if its static water contact angle is >90°. Without wishing to be bound by theory, it is postulated that using a hydrophobic substrate leads to improved adhesion between the cells and the substrate. Without wishing to be bound by theory, using a hydrophobic substrate may also prevent solutions of the flavonol and anthocyanidin mono-glycoside pigments from spreading unduly, which ensures that the pigments are retained over the immobilised cells on the substrates. If undue spreading is observed using one type of substrate, it may be beneficial to repeat the method using a more hydrophobic substrate.

Substrates can be made hydrophobic by applying suitable silane coatings, such as coatings of 3-aminopropyl trimethoxysilane (CAS 13822-56-5). Glass substrates having a high sodium content may be suitable hydrophobic substrates.

After transferring the cells to the transparent substrate the cells are dried to immobilise them on the substrate before being treated with the pigments. Drying should take place immediately after transfer of the cells to the substrate. Suitable drying conditions are in air under ambient conditions for about 10 to 30 minutes, typically at least about 20 minutes. The transparent substrate comprising the dried cells may then be stored until the treatment with the flavonol and anthocyanidin mono-glycoside pigments is carried out. Storage is preferably under ambient conditions and in a dark environment.

The difference in nuclear staining between cells from subjects who have and do not have endometriosis is more apparent when using cells which have been recently obtained from the subjects. Thus, it is preferred that the treatment with the pigments in the invention is carried out within 2 months after drying of the cells on the substrate. Preferably, the treatment with the pigments in the invention is carried out within 1 month, more preferably within 1 day, more preferably within 3 hours, most preferably within 1 hour, after drying of the cells on the substrate. Preferably, the samples are treated with the flavonol and anthocyanidin mono-glycoside pigments immediately after drying.

Alternatively, when cells are collected in the form of a saliva sample, the saliva sample can be frozen and stored for longer periods of time. Frozen saliva samples can be thawed and then applied to the transparent substrate. For example, saliva can be collected into an ampule by drooling into the ampule and the collected saliva sample can be stored in a freezer at −80° C.

Thus, in other preferred embodiments, when cells are collected in the form of a saliva sample, the saliva sample may be stored under cold conditions in a freezer (e.g. at a temperature of from −5 to −90° C., such as −10 to −85° C.). Suitably, the saliva sample may be frozen within 2 months, preferably within 1 month, more preferably within 1 day, more preferably within 3 hours, more preferably within 1 hour after the sample has been obtained from the subject. Most preferably, the saliva sample is frozen immediately after the sample has been obtained from the subject. The saliva sample may be stored under the cold conditions for periods of up to about 2 years, such as up to about 1 year.

Suitably, the cells from the saliva sample may be treated with the pigments in accordance with the invention within a week after the sample has thawed such as within 3 hours after the sample has thawed, such as within 1 hour after the sample has thawed.

Without wishing to be bound by theory, the inventor postulates that the cells may disintegrate if left on the substrate for long periods of time before being treated with the pigments in the invention, particularly if the cells are kept under humid conditions. It is therefore recommended to store transparent substrates on which the cells have been immobilised with a desiccating material. Disintegration of cells may interfere with the staining behaviour of the cells when treated with the pigments. Optionally, the dried sample can be observed prior to treatment with the pigments in order to assess cell quality.

The presence or absence of staining of the cell nuclei can be observed visually, for example using an optical microscope, preferably using bright-field (BF) microscopy. Suitable optical microscopes include those providing 100× to 400× magnification and are available from, for example, Nikon. Suitably, the magnified samples are then photographed using a digital camera to provide a record of the test result.

Cells including their nuclei can be observed visually (for example using an optical microscope as set out above) before treatment with the pigments of the invention. In this way, the appearance of a given cell can be compared before and after treatment with the pigments in accordance with the invention in order to facilitate identification of nuclear staining. Alternatively, a cell which has been treated with the pigments in accordance with the invention can be compared to a different cell from the same subject which has not been treated with the pigments in order to facilitate identification of nuclear staining. Conveniently, only a portion of the cells in a sample may be treated with the pigments so that a side by side comparison of treated and untreated cells can be made.

Only cells with nuclei show differences in staining behaviour in the methods of the invention. When observing samples which have been treated with the pigments, care should be taken to identify cells containing nuclei as not all cells in a sample will necessarily contain nuclei. For example, a sample collected from the mouth will contain mucosal cells in different stages of the cell life cycle. Some cells will have nuclei and some may be cornified, or have lost their nuclei. In an oral sample, there may be many cells without nuclei as the mouth is constantly shedding dead cells that have lost these structures. Optionally, the dried sample can be observed prior to treatment with the pigments to ensure that the sample includes cells containing nuclei. If no cell nuclei are visible in a sample, another sample should be taken from the subject for testing. Mucosal epithelial cells, such as oral epithelial cells, go through a 25-day cornification process that is regulated by certain estrogen receptors and epidermal growth factor that play a role in managing cell morphology changes with the menstrual cycle. In subjects without endometriosis fewer cells are cornified during the follicular phase. In the invention, is therefore preferred to use cells that have been obtained during the follicular phase.

Preferably, the subject is not taking any birth control medication when the cells are obtained from the subject.

### Anthocyanidin Mono-Glycoside

The methods of the invention use an anthocyanidin mono-glycoside pigment which has at least two OH groups on the B ring of the anthocyanidin. Anthocyanidin mono-glycosides are anthocyanins containing one glycoside unit.

Anthocyanins are glycosides of flavylium salts. Each anthocyanin thus comprises three component parts: a hydroxylated core (the aglycone derived from the anthocyanidin), a saccharide unit (glycoside) and a counter ion. Anthocyanins are naturally occurring pigments present in many flowers and fruit. Individual anthocyanins, including anthocyanidin mono-glycosides, are available commercially, for example from Sigma Aldrich or from Polyphenols Laboratories AS, Sandnes, Norway.

Use of anthocyanidin mono-glycoside pigments rather than anthocyanidin di-glycoside pigments has been found to be necessary for the nuclei in cells from women not suffering from endometriosis to appear darker than the surrounding part(s) of the cell when carrying out the methods of the invention. Without wishing to be bound by theory, it is believed that the anthocyanidin mono-glycoside pigments can potentially cross the lipid membrane of a cell and hence reach the cell nucleus. Differences in the composition of the cell membranes in women with and without endometriosis is postulated to lead to differences in the transfer of the anthocyanidin mono-glycoside pigments into the cells and hence the difference in nuclear staining/coloration which is observed. In contrast, it has been observed that anthocyanidin di-glycoside pigments may simply stain the surface of the cell and cannot be used to differentiate between cells from subjects with and without endometriosis.

It has also been found that anthocyanidin mono-glycosides lacking at least two OH groups on the B ring do not exhibit differences in nuclear staining behaviour in cells from women with and without endometriosis. Hence these pigments cannot be used in the methods of the invention to differentiate between the presence or absence of endometriosis in a subject when comparing staining responses in cell nuclei.

Anthocyanidin mono-glycoside pigments useful in the methods of the invention possess the following structure (Formula I):

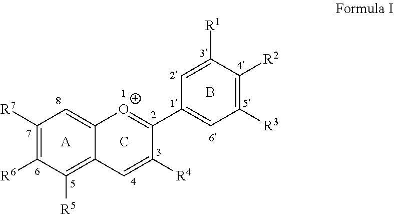

wherein:
   one of R⁴ and R⁵ denotes O-glycoside and the other denotes H or OH;
   R¹, R² and R³ each independently denote H, OH or C₁₋₄ alkoxy, provided that at least two of R¹, R² and R³ denote OH;
   R⁶ denotes H, OH or C₁₋₄ alkoxy; and
   R⁷ denotes H, OH or C₁₋₄ alkoxy.
   The anthocyanidin mono-glycoside, such as compounds of Formula I, will typically be present in combination with a suitable counterion, for example chloride, acetate or citrate, preferably chloride.

In the above definitions, preferred C₁₋₄ alkoxy groups are methoxy or ethoxy, more preferably methoxy.

Preferably, the B ring in the compound of Formula I comprises at least two adjacent OH groups (i.e. preferably R¹ and R² are both OH, or R² and R³ are both OH, or each of R¹, R² and R³ is OH). More, preferably, R¹ and R² denote OH and R³ denotes H; or R² and R³ denote OH and R¹ denotes H; or each of R¹, R² and R³ denote OH.

Preferably, R⁴ denotes O-glycoside.

Also preferably, R⁵ denotes OH.

Also preferably, R⁶ denotes H.

Also preferably, R⁷ denotes OH.

The definitions of R¹, R², R³, R⁵, R⁶ and R⁷ in Formula I for the cores of some suitable naturally occurring anthocyanins for use in the invention are as follows:

| Aglycone | R¹ | R² | R³ | R⁵ | R⁶ | R⁷ |
|---|---|---|---|---|---|---|
| Cyanidin | OH | OH | H | OH | H | OH |
| Delphinidin | OH | OH | OH | OH | H | OH |
| Petunidin | OCH₃ | OH | OH | OH | H | OH |

R⁴ is an O-glycoside in these naturally occurring anthocyanins.

Anthocyanidin mono-glycosides wherein R¹, R², R³, R⁵, R⁶ and R⁷ are defined as in the table above and R⁴ is O-glycoside (preferably, O-glucoside) may be used in the invention.

The glycoside may be derived from a mono- or disaccharide. Derived from in this context means that the anthocyanidin mono-glycoside is a reaction product of the anthocyanidin with the mono- or disaccharide. Examples of suitable monosaccharides include glucose, galactose, fructose, xylose, rhamnose and arabinose. An example of a suitable disaccharide is rutinose (i.e. 6-rhamnosyl-glucose). Monosaccharides, and in particular glucose, are preferred. Particularly preferred sugars include D-glucose, D-galactose, D-arabinose and 6-L-rhamnosyl-D-glucose, more preferably D-glucose.

A preferred anthocyanin for use in the invention is cyanidin-3-glucoside. The cyanidin-3-glucoside may comprise any suitable counterion, and the counterion is preferably chloride.

### Flavonol

The methods of the invention use a flavonol pigment. Flavonols are compounds with the following core structure (Formula II):

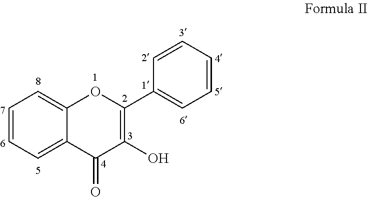 [SMILES: O=c1c(O)c(-c2ccccc2)oc2ccccc12]

The 5, 6, 7, 8, 2′, 3′, 4′, 5′, and 6′ positions in Formula II may independently be substituted with OH or C₁₋₄ alkoxy. Preferred C₁₋₄ alkoxy groups are methoxy or ethoxy, more preferably methoxy. Preferably, no more than 6 of the 5, 6, 7, 8, 2′, 3′, 4′, 5′, and 6′ positions are substituted, more preferably no more than 3 of these positions are substituted.

The structures of some common flavonols which may be of use in the present invention are shown in the table below, where the position numbering relates to Formula II.

| | Position | | | | | | | | |
|---|---|---|---|---|---|---|---|---|---|
| Name | 5 | 6 | 7 | 8 | 2′ | 3′ | 4′ | 5′ | 6′ |
| 3- | H | H | H | H | H | H | H | H | H |
| hydroxyflavone | | | | | | | | | |
| Azaleatin | OCH₃ | H | OH | H | H | H | OH | OH | H |
| Fisetin | H | H | OH | H | H | OH | OH | H | H |
| Galangin | OH | H | OH | H | H | H | H | H | H |
| Gossypetin | OH | H | OH | OH | H | OH | OH | H | H |
| Kaempferide | OH | H | OH | H | H | H | OCH₃ | H | H |
| Kaempferol | OH | H | OH | H | H | H | OH | H | H |
| Isorhamnetin | OH | H | OH | H | H | OCH₃ | OH | H | H |
| Morin | OH | H | OH | H | OH | H | OH | H | H |
| Myricetin | OH | H | OH | H | H | OH | OH | OH | H |
| Natsudaidain | OCH₃ | OCH₃ | OCH₃ | OCH₃ | H | H | OCH₃ | OCH₃ | H |
| Pachypodol | OH | H | OCH₃ | H | H | OCH₃ | OH | H | H |
| Quercetin | OH | H | OH | H | H | OH | OH | H | H |
| Rhamnazin | OH | H | OCH₃ | H | H | OCH₃ | OH | H | H |
| Rhamnetin | OH | H | OCH₃ | H | H | OH | OH | H | H |

A preferred flavonol pigment for use in the methods of the invention is quercetin.

Flavonol pigments are available commercially, for example from Sigma Aldrich.

The methods of the invention utilize a flavonol pigment and an anthocyanidin mono-glycoside pigment. Preferably the flavonol is selected from quercetin, kaempferol, myricetin, fisetin, galangin, isorhamnetin, pachypodol, or rhamnazin and the anthocyanidin mono-glycoside is cyanidin-3-glucoside. A particularly preferred method utilizes quercetin and cyanidin-3-glucoside.

Methods of the invention comprise treating (i.e. contacting) cells with the pigments sequentially. It is important that the cells are not treated with the flavonol pigment and anthocyanidin mono-glycoside pigment simultaneously, i.e. that the pigments are not applied to the cells together, and that the flavonol pigment is applied first followed by the anthocyanidin mono-glycoside pigment. If the flavonol pigment and anthocyanidin mono-glycoside pigment are applied simultaneously, no difference in staining behavior is seen for cells from women with and without endometriosis. If the anthocyanidin mono-glycoside pigment is applied first, this leads to staining of only the outside of the cell.

In the methods of the invention, the cells are first treated with the flavonol pigment and later treated with the anthocyanidin mono-glycoside pigment. The samples should be dried after application of the flavonol pigment before the anthocyanidin mono-glycoside pigment is applied. Preferably, there are no intermediate steps, other than drying, between the steps of treating with the flavonol pigment and treating with the anthocyanidin mono-glycoside pigment.

In the methods of the invention, the cells are preferably treated with solutions of the flavonol and the anthocyanidin mono-glycoside. Suitable solvents for both the flavonol and the anthocyanidin mono-glycosides are volatile and include methanol, ethanol, acetone and toluene, preferably methanol. Suitable concentrations for the solutions are 6×10⁻³ molar to 0.1×10⁻³ molar, preferably 2×10⁻³ molar to 0.5×10⁻³ molar, most preferably 1×10⁻³ molar.

The ratio (mol/mol) of the amount of flavonol to the amount of anthocyanidin mono-glycoside added to the cells may be about 2:1 to about 1:2, preferably about 1:1. Preferably, the cells are treated with substantially the same amounts (in moles) of the flavonol and the anthocyanidin mono-glycoside.

Suitable amounts of the solutions of flavonol and anthocyanidin mono-glycoside for application to the cells on the substrate are in the region of about 0.5 to about 30 microliters, preferably around 1 to 25 microliters, more preferably about 5 to about 20 microliters, for example about 10 microliters or about 20 microliters. In other preferred cases, the amounts of the solutions are about 1 to 15 microliters.

After application, the solvent(s) is/are allowed to evaporate. Evaporation of the solvents preferably occurs under ambient conditions. If the flavonol and anthocyanidin mono-glycoside are applied in solution, the flavonol solution applied first is preferably allowed to dry, for example for about 10-30 minutes such as about 20 minutes, before the second solution is applied.

Similarly, after application the anthocyanidin mono-glycoside solution is preferably allowed to dry, for example for about 10-30 minutes such as about 20 minutes, before inspecting the samples.

Once the cells have been treated with the pigments, the treated samples can be viewed immediately. Alternatively, the treated samples can be stored for up to about a year before being viewed. Storage is preferably under ambient conditions and in a dark environment.

In some preferred cases, the cells are treated by the subject, i.e. preparation method A is carried out by the subject, optionally using a kit of the invention. The subject may then send the treated sample(s) to another party for observation/analysis, e.g. according to screening/diagnosis method A. Preparation method A may therefore conveniently be implemented using a home test kit. Observation and analysis is conveniently carried out in a laboratory equipped with a suitable microscope for viewing the samples.

### Kits

The invention also provides a kit for preparing samples (or cells) for screening for, or diagnosing, endometriosis, the kit comprising a flavonol pigment, preferably in the form of a solution as described above, and an anthocyanidin mono-glycoside pigment which has at least two OH groups on the B ring, preferably in the form of a solution as described above. The kit preferably comprises instructions to direct the user to carry out a method of the invention, such as preparation method A of the invention. The kit preferably further comprises an oral swab, such as a cellulose stick, and one or more transparent substrates, such as a glass or clear plastic slide. The instructions preferably further comprise directions on how to extract the cells using the swab and/or how to immobilise the cells onto the transparent substrate.

This can be done by rolling the swab on the mucosal tissue for about 2 seconds followed by swiping the exposed swab in one direction on the transparent surface and then letting the sample dry in ambient conditions for at least 20 minutes.

Preferably, the methods of the invention do not comprise treating the cells with any apatite compounds.

Preferably, the methods of the invention do not comprise treating the cells with hydroxyapatite.

If the screening/diagnosis method of the invention returns a positive result (i.e. if cells without nuclear staining are observed), a diagnosis of endometriosis may be confirmed by laparoscopy, ultrasound and/or MRI imaging.

The screening methods of the invention are non-invasive and relatively low cost and so may be used to screen subjects whether or not it is suspected that the subject may have endometriosis. However, typically the subject may be displaying symptoms of endometriosis.

If the screening/diagnosis method of the invention returns a positive result for endometriosis, the methods of the invention may further comprise treating the subject using pain relief medication and/or a treatment for endometriosis, for example a hormone-based treatment or treatment with a GnRH agonist or birth control pills to control oestrogen levels.

### EXAMPLES

Cells were observed using bright-field (BF) microscopy.

### Preparation of Pigment Solution for Pigment 1

A 1×10⁻³ molar solution of quercetin was prepared by mixing 1.21 mg of quercetin (Sigma Aldrich) with 4 ml of methanol in a screw top tube and agitating the mixture until all the pigment was dissolved. The solution was stored in the dark under ambient conditions until required.

### Preparation of Pigment Solution for Pigment 2

A 1×10⁻³ molar solution of cyanidin-3-glucoside was prepared by mixing 1.94 mg of cyanidin-3-glucoside chloride (Polyphenols AS, Sandnes, Norway) with 4 ml of methanol in a screw top tube and agitating the mixture until all the pigment was dissolved. The solution was stored in the dark under ambient conditions until required.

### Sample Preparation

Cheek cells were harvested from subjects by swabbing the inner cheek in the soft palate area of the mouth for about 2-10 seconds using a cellulose stick. Cells from a subject were transferred to a glass slide by sliding the cellulose stick in one direction over the slide surface. The glass slides were then allowed to air dry under ambient conditions for about 20 minutes to immobilise the cells on the slides. The untreated samples were then observed using an optical microscope at 400× magnification to confirm that cells were present in the sample. The cells on the glass slides were then treated with pigments as set out below.

### Example 1 (Comparative)

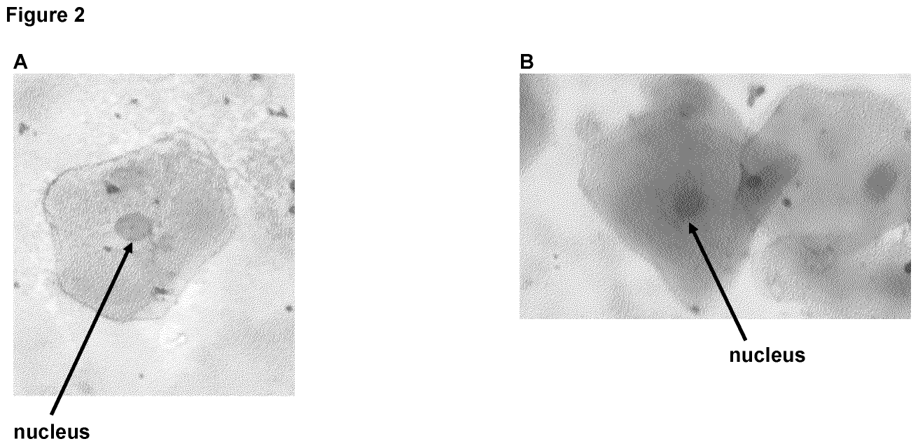

Samples harvested according to the procedure set out above were treated with a 2% solution of methylene blue. As illustrated in FIG. 2, methylene blue pigment staining is non-selective for endometriosis as strong nuclear staining is seen in the cells from subjects who were known not to have endometriosis as well as those who were known to have endometriosis.

### Example 2 (Comparative)

Samples (cells on glass slides) harvested according to the procedure set out above were treated with 10 microliters of quercetin (concentration 1×10⁻³ molar in methanol) using a pipette. The samples were allowed to dry under ambient conditions for 20 minutes. The samples were then treated with 10 microliters of malvidin-3,5-diglucoside (concentration 1×10⁻³ molar in methanol) using a pipette and then allowed to dry under ambient conditions for 20 minutes. The cells were then photographed at 400× magnification using a Nikon bifocal optical microscope.

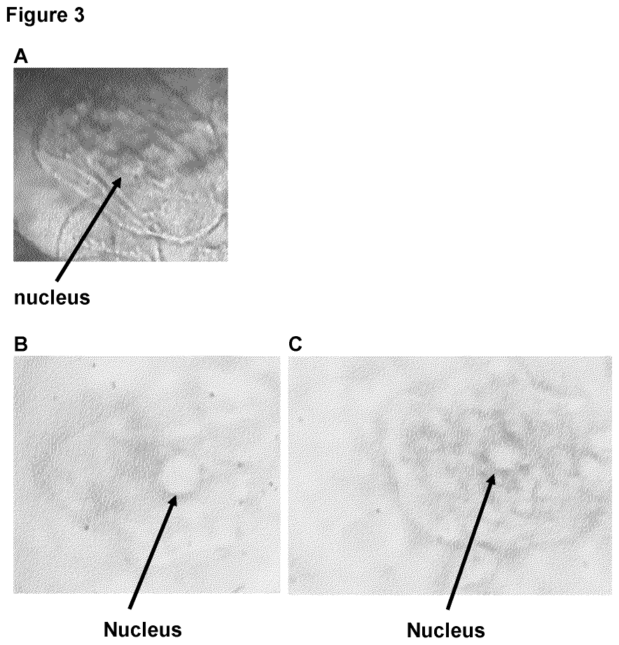

As illustrated in FIG. 3, treatment with quercetin and then malvidin-3,5-diglucoside only stained the cell surface. Visual examination of the cells showed no differentiation in colour intensity between the cell nucleus and surrounding parts of the cell. (FIGS. 3A-3C). Use of malvidin-3,5-diglucoside cannot therefore distinguish between cells from subjects with or without endometriosis in this method.

### Example 3

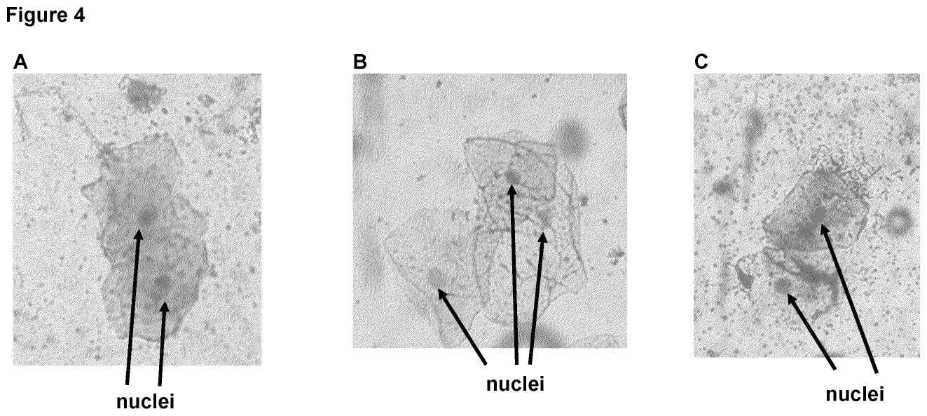

For the samples of FIGS. 4, 6 and 7, samples on glass slides prepared according to the procedure set out above were treated with 10 microliters of the pigment 1 solution using a pipette. The samples were allowed to dry under ambient conditions for 20 minutes. The samples were then treated with 10 microliters of the pigment 2 solution using a pipette and allowed to dry under ambient conditions for 20 minutes.

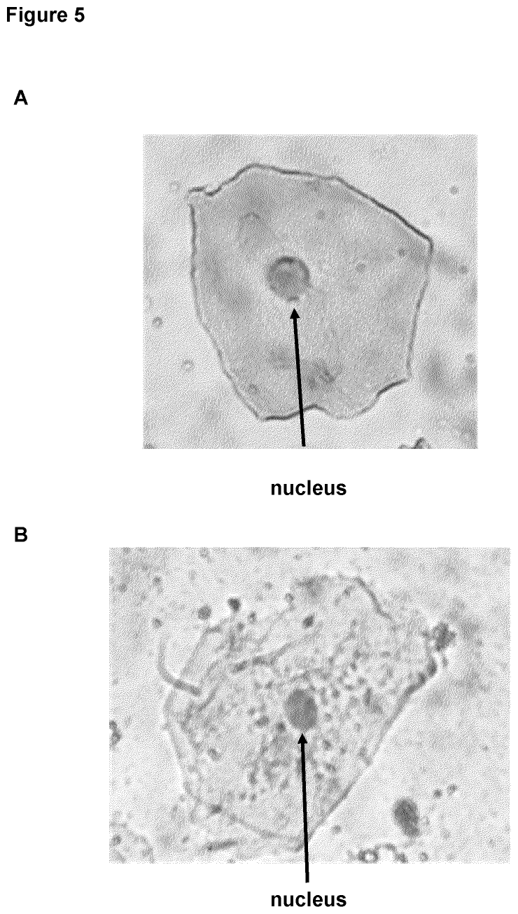

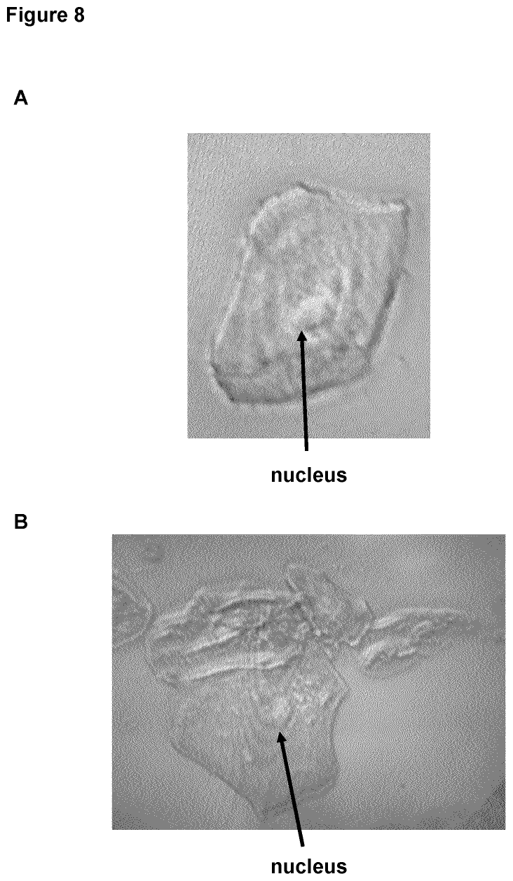

An analogous procedure was used for the samples of FIGS. 5 and 8, except 20 microliters of the pigment 1 and 2 solutions were used. The cells photographed in FIGS. 5 and 8 were taken from subjects during the follicular phase. The cells photographed in FIGS. 8A and 8B were taken on days 2 and 3 of the subject's menstrual cycle (i.e. during the early follicular phase).

The treated samples were viewed under a microscope to identify cells containing nuclei. The cells were then photographed at 400× magnification using a Nikon bifocal optical microscope.

Photographs of treated cells from the subjects who were known not have endometriosis are shown in FIGS. 4A-C and 5. As shown in FIGS. 4 and 5, the treatment led to a significant contrast in staining between the nucleus and the surrounding parts of the cell. The nuclei appear darker than the surrounding parts of the cell.

Photographs of treated cells from the subjects who were known to have endometriosis are shown in FIGS. 6A-6D, 7 and 8. As shown in FIGS. 6-8, the cell nuclei did not appear darker than the surrounding parts of the cell. The preparation method of the invention therefore allows the cells from subjects who have endometriosis to be distinguished visually from those who do not have endometriosis. Also shown in FIG. 7A are dark spots which were artefacts formed outside the cells during sample preparation. They are not diagnostic for the presence or absence of endometriosis.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. The specification describes specific examples to accomplish a more general goal that may be accomplished in another way.

1. An in vitro method for preparing cells such that they are suitable for use in screening for, or diagnosis of, endometriosis in a human female subject, the method comprising:
sequentially treating mucosal epithelial cells which have been obtained from the female subject and immobilised on a transparent substrate with i) a flavonol pigment and ii) an anthocyanidin mono-glycoside pigment which has at least two OH groups on the B ring of the anthocyanidin, in that order.

2. The method of claim 1, wherein the anthocyanidin mono-glycoside is a compound of Formula I: 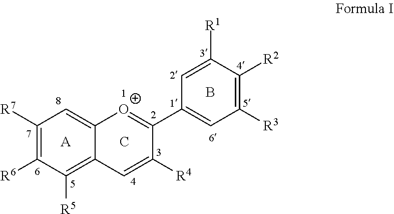
wherein:
one of R⁴ and R⁵ denotes O-glycoside and the other denotes H or OH;
R¹, R² and R³ each independently denote H, OH or C₁₋₄ alkoxy, provided that at least two of R¹, R² and R³ denote OH;
R⁶ denotes H, OH or C₁₋₄ alkoxy; and
R⁷ denotes H, OH or C₁₋₄ alkoxy.

3. The method of claim 1, wherein the anthocyanidin mono-glycoside pigment is an anthocyanidin mono-glucoside.

4. The method of claim 3, wherein the anthocyanidin mono-glycoside is cyanidin-3-glucoside.

5. The method of claim 1, wherein the flavonol is a compound of Formula II: 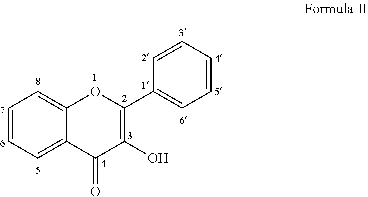 [SMILES: O=c1c(O)c(-c2ccccc2)oc2ccccc12]
wherein the 5, 6, 7, 8, 2′, 3′, 4′, 5′, and 6′ positions in Formula II may independently be substituted with OH or C₁₋₄ alkoxy.

6. The method of claim 5, wherein the flavonol is quercetin.

7. The method of claim 1, wherein the ratio (mol/mol) of the flavonol to the anthocyanidin mono-glycoside is about 1:1.

8. The method of claim 1, wherein the method does not comprise treating the cells with any apatite compound(s).

9. The method of claim 1, wherein the mucosal epithelial cells have been obtained from the female subject during the follicular phase of the menstrual cycle.

10. The method of claim 1, wherein the mucosal epithelial cells have been obtained from the female subject during the early follicular phase, wherein the early follicular phase is defined as day 1 to day 8 of the of the subject's menstrual cycle, wherein the first day of menstrual bleeding is considered day 1 of the menstrual cycle.

11. The method of claim 1, wherein the transparent substrate is hydrophobic.

12. A treated mucosal epithelial cell obtained by the method of claim 1.

13. An in vitro method of screening for, or diagnosing, endometriosis in a human female subject, the method comprising:
observing whether or not the nuclei of one or more cells obtained by the method of claim 1 appear to be darker than the surrounding part(s) of the cell(s).

14. (canceled)

15. The method of claim 13, wherein if the cell nuclei do not appear darker than the surrounding part(s) of the cell(s) this is indicative that the subject has endometriosis.

16. The method of claim 13, wherein if the cell nuclei appear darker than the surrounding part(s) of the cell(s), this is indicative that the subject does not have endometriosis.

17. The method of claim 13, wherein the observing is carried out using bright field microscopy.

18. A kit for preparing samples for screening for, or diagnosing, endometriosis comprising a flavonol pigment and an anthocyanidin mono-glycoside pigment which has at least two OH groups on the B ring of the anthocyanidin.

19. The kit of claim 18, further comprising instructions to direct the user to carry out an in vitro method for preparing cells such that they are suitable for use in screening for, or diagnosis of, endometriosis in a human female subject, the method comprising:
sequentially treating mucosal epithelial cells which have been obtained from the female subject and immobilised on a transparent substrate with i) the flavonol pigment and ii) the anthocyanidin mono-glycoside pigment, in that order.
